(19) United States
(12) Patent Application Publication
(10) Pub. No.: US 2026/0325855 A1
(43) Pub. Date: Oct. 22, 2026

(54) APPARATUS AND METHOD FOR CANCER TREATMENT USING ELECTRIC FIELD AND ULTRASOUND

(71) Applicant: Electronics and Telecommunications Research Institute, Daejeon (KR)

(72) Inventors: Hyung Ju PARK, Daejeon (KR); Soo Jun PARK, Daejeon (KR); Chul HUH, Daejeon (KR)

(73) Assignee: Electronics and Telecommunications Research Institute, Daejeon (KR)

(21) Appl. No.: 19/549,300

(22) Filed: Feb. 25, 2026

(30) Foreign Application Priority Data
Apr. 18, 2025 (KR) 10-2025-0050935

(51) Int. Cl. A61N 1/40; A61K 41/00; A61N 7/00; B06B 1/06

(52) U.S. Cl. CPC A61N 1/40; A61K 41/0052; A61N 7/00; B06B 1/0622

(57) ABSTRACT

A cancer treatment apparatus is provided. The cancer treatment apparatus comprises, a signal generator, and a first electrode array and a second electrode array electrically connected to the signal generator and facing each other with a target region in which cancer cells are present disposed therebetween. The signal generator generates an electric field and ultrasound between the first electrode array and the second electrode array. Each of the first electrode array and the second electrode array includes electric-field electrodes and ultrasound electrodes.

### CROSS-REFERENCE TO RELATED APPLICATIONS

This application claims priority under 35 U.S.C. § 119 to Korean Patent Application No. 10-2025-0050935 filed on Apr. 18, 2025, in the Korean Intellectual Property Office, the disclosures of which are incorporated by reference herein in their entireties.

### BACKGROUND

The present disclosure relates to a cancer treatment technology using an electric field, and more particularly, to a cancer treatment apparatus and an operation method thereof using a ferroelectric nanoparticle probe, an electrode array, an electric field, and ultrasound.

When cancer occurs, general treatment methods currently include surgery, chemotherapy, and radiation therapy. However, the currently used cancer treatment methods have problems in that damage to normal tissues increases a patient’s pain, and inconvenience of hospitalization or outpatient treatment lowers the quality of life of cancer patients. To overcome these problems, efforts have been made to develop an electronic system that affects cellular functions in warm-blooded mammals by using a low-energy frequency-controlled signal generator, and to develop the electronic system into a cancer treatment medical apparatus.

For cancer patients with high mortality rates, such as those with brain tumors for which therapeutic effects have not been high with conventional cancer treatment technologies, an electromagnetic field-based cancer treatment apparatus not only improves therapeutic effects but also improves patients’ quality of life, thereby increasing utility in the field of alternative cancer treatment technologies. Nevertheless, there are still many insufficient aspects in research on electromagnetic field-based cancer treatment apparatuses and cancer treatment methods using the same, which require long-term exposure to electromagnetic fields and are intended to reduce damage to normal cells in which cell division actively occurs. Accordingly, research in this field is being actively conducted.

### SUMMARY

An object of the present invention is to provide a cancer treatment apparatus and an operation method thereof that combine an electric field and high-intensity focused ultrasound to enhance delivery efficiency of ferroelectric nanoparticle probes, which are intelligent drugs, improve permeability through the blood-brain barrier (BBB), and maximize therapeutic effects on cancer cells.

According to an embodiment of the present disclosure, a cancer treatment apparatus includes a signal generator; and a first electrode array and a second electrode array electrically connected to the signal generator and facing each other with a target region containing cancer cells disposed therebetween. The signal generator generates an electric field and ultrasound between the first electrode array and the second electrode array, and each of the first electrode array and the second electrode array includes electric-field electrodes and ultrasound electrodes.

In one embodiment, the signal generator includes an electric-field generator configured to apply an AC (Alternating Current) voltage between the first electrode array and the second electrode array; and an ultrasound generator configured to apply an AC voltage having a specific frequency between the first electrode array and the second electrode array.

In one embodiment, the signal generator includes a controller configured to adjust application amounts of the electric field and the ultrasound based on an administration time of a cancer treatment drug.

In one embodiment, the first electrode array is configured such that one electric-field electrode is disposed at a center and a plurality of electric-field electrodes are circularly arranged around the center, and ultrasound electrodes are radially disposed between the electric-field electrodes, and the second electrode array has the same configuration.

In one embodiment, each ultrasound electrode included in the first electrode array and the second electrode array includes a metal plate configured to transmit an electrical signal, a piezoelectric element configured to vibrate by the electrical signal, and a vibration plate configured to protect the ultrasound electrode and the piezoelectric element.

According to another embodiment of the present disclosure, a cancer treatment apparatus includes a signal generator; and a first electrode array and a second electrode array electrically connected to the signal generator and facing each other with a target region containing cancer cells disposed therebetween. The signal generator is configured to generate an electric field between the first electrode array and the second electrode array to change an orientation of nanoparticle probes within cancer cells, and to generate ultrasound between the first electrode array and the second electrode array to enhance permeability of the nanoparticle probes through a blood-brain barrier. Each of the first electrode array and the second electrode array includes electric-field electrodes and ultrasound electrodes.

In one embodiment, the signal generator includes an electric-field generator configured to apply an AC (Alternating Current) voltage between the first electrode array and the second electrode array; and an ultrasound generator configured to apply an AC voltage having a specific frequency between the first electrode array and the second electrode array.

In one embodiment, the signal generator includes a controller configured to adjust application amounts of the electric field and the ultrasound based on an administration time of the nanoparticle probes.

In one embodiment, the first electrode array is configured such that one electric-field electrode is disposed at a center and a plurality of electric-field electrodes are circularly arranged around the center, and ultrasound electrodes are radially disposed between the electric-field electrodes, and the second electrode array has the same configuration.

In one embodiment, each ultrasound electrode included in the first electrode array and the second electrode array includes a metal plate configured to transmit an electrical signal, a piezoelectric element configured to vibrate by the electrical signal, and a vibration plate configured to protect the ultrasound electrode and the piezoelectric element.

In one embodiment, the nanoparticle probe includes a ferroelectric particle having a polygonal shape, a non-dielectric insulating layer coated on the ferroelectric particle, and a plurality of biomarkers attached to the non-dielectric insulating layer, wherein the biomarkers target cancer cells.

In one embodiment, the biomarkers of the nanoparticle probe include at least one of transferrin and folate.

In one embodiment, the ferroelectric particle has a diameter greater than 1 nm and less than or equal to 100 nm, and the ferroelectric particle is formed of BaTiO₃.

In one embodiment, the non-dielectric insulating layer includes at least one of silicon oxide (SiO₂), aluminum oxide (Al₂O₃), and silicon nitride (Si₃N₄).

According to one embodiment of the present disclosure, a method of operating a cancer treatment apparatus using ferroelectric nanoparticle probes includes applying a voltage to a first electrode array and a second electrode array by a signal generator included in the cancer treatment apparatus; and irradiating a target region containing cancer cells with an electric field and ultrasound by the first electrode array and the second electrode array.

In one embodiment, the method further includes adjusting an intensity of the voltage based on an administration time of the ferroelectric nanoparticle probes by a controller included in the cancer treatment apparatus.

### BRIEF DESCRIPTION OF THE FIGURES

The above and other objects and features of the present disclosure will become apparent by describing in detail embodiments thereof with reference to the accompanying drawings.

FIG. 1 illustrates a configuration of a cancer treatment apparatus according to an embodiment of the present disclosure.

FIG. 2 is a conceptual diagram illustrating a first electrode array, a second electrode array, and an affected region according to some embodiments.

FIG. 3 illustrates an arrangement structure of electric-field electrodes and ultrasound electrodes included in the first electrode array and the second electrode array of FIGS. 1 and 2.

FIG. 4 illustrates a structure of a first ultrasound electrode included in the first electrode array of FIGS. 1 and 2.

FIG. 5 illustrates a case in which the cancer treatment apparatus according to an embodiment of the present disclosure is used together with nanoparticle probes.

FIG. 6 illustrates a configuration of a nanoparticle probe.

FIG. 7 is a comparison diagram between a comparative example without a silica layer (SiO₂) and an experimental example with a silica layer (SiO₂).

FIG. 8 is a graph illustrating an electric-field intensity in the experimental example with a silica layer (SiO₂) compared to the comparative example without a silica layer (SiO₂).

FIG. 9 is a schematic diagram illustrating a strategy for enhancing electric-field-based cancer treatment efficiency by adjusting an ultrasound intensity according to an administration amount of a drug including nanoparticle probes.

FIG. 10 illustrates an example of use of the cancer treatment apparatus according to the present disclosure.

FIG. 11 is a flowchart illustrating an operation method of the cancer treatment apparatus according to the present disclosure.

### DETAILED DESCRIPTION

Hereinafter, embodiments of the present disclosure will be described clearly and in detail such that a person having ordinary skill in the art to which the present disclosure pertains can readily practice the present disclosure.

Terms such as “unit” and “module” used herein, or functional blocks illustrated in the drawings, may be implemented in the form of software, hardware, or a combination thereof. In the following description, detailed descriptions of well-known components will be omitted to clearly explain the technical spirit of the present invention.

As used herein, expressions such as “A or B,” “at least one of A and B,” “at least one of A or B,” “A, B, or C,” “at least one of A, B, and C,” and “at least one of A, B, or C” may include any one of the listed items or any combination thereof.

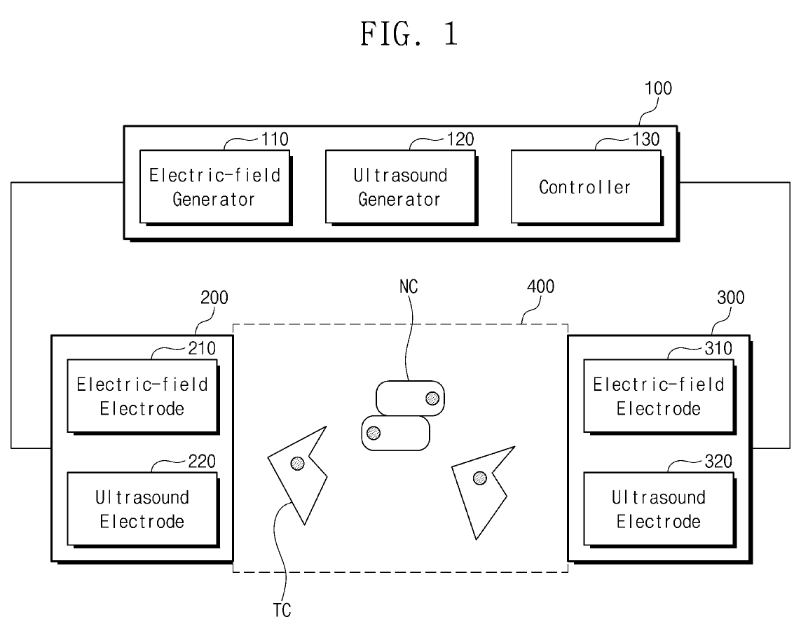

FIG. 1 illustrates a configuration of a cancer treatment apparatus according to an embodiment of the present disclosure. Referring to FIG. 1, the cancer treatment apparatus may include a signal generator 100, a first electrode array 200, and a second electrode array 300.

The signal generator 100 may include an electric-field generator 110, an ultrasound generator 120, and a controller 130.

In one embodiment, the signal generator 100 may be electrically connected to the first electrode array 200 and the second electrode array 300. The signal generator 100 may be connected to the electrode arrays based on wires, a flexible printed circuit (FPC), or a printed circuit board (PCB) pattern. The signal generator 100 may be connected to the electrode arrays based on a coaxial cable having an impedance of 50 Ω for high-frequency signal transmission.

In one embodiment, the electric-field generator 110 may apply an AC (Alternating Current) voltage between the first electrode array 200 and the second electrode array 300. The electric-field generator 110 may control an intensity of an electric field generated between the first electrode array 200 and the second electrode array 300 based on adjusting a magnitude of AC voltage.

In one embodiment, the ultrasound generator 120 may apply an AC voltage having a specific frequency between the first electrode array 200 and the second electrode array 300, which include piezoelectric elements. The ultrasound generator 120 may control an intensity of ultrasound generated between the first electrode array 200 and the second electrode array 300 based on adjusting a magnitude of the AC voltage having the specific frequency.

In one embodiment, the controller 130 may control individual frequencies and intensities of the electric field and the ultrasound applied between the first electrode array 200 and the second electrode array 300. The controller 130 may adjust the intensities of the electric field and the ultrasound based on an administration time of a cancer treatment drug.

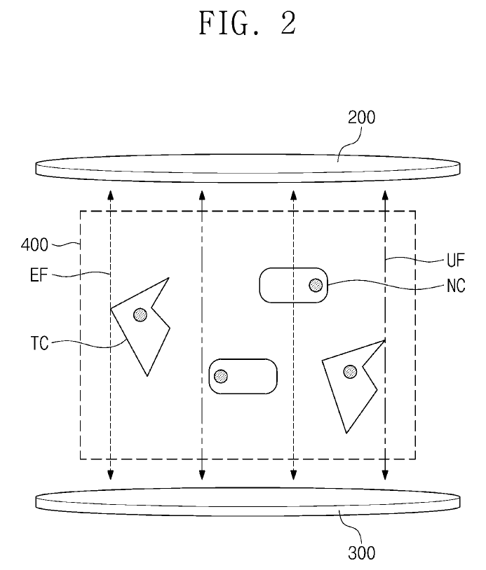

Referring to FIGS. 1 and 2, the first electrode array 200 and the second electrode array 300 may be disposed to be spaced apart from each other with an affected region 400 interposed therebetween. The affected region 400 may be an area in which cancer cells TC are present, and may also be referred to as a target region. Normal cells NC may also be present in the affected region 400. The affected region 400 may correspond, for example, to any one region of a body such as a brain in which cancer cells TC are present.

In one embodiment, at least a portion of the first electrode array 200 and at least a portion of the second electrode array 300 may face each other.

In one embodiment, the first electrode array 200 may include a first electric-field electrode 210 and a first ultrasound electrode 220, and the second electrode array 300 may include a second electric-field electrode 310 and a second ultrasound electrode 320. As illustrated in FIG. 2, the first electrode array 200, the second electrode array 300, and the first electric-field electrode 210, the first ultrasound electrode 220, the second electric-field electrode 310, and the second ultrasound electrode 320 included therein may each have a disk shape. Each of the first electric-field electrode 210, the first ultrasound electrode 220, the second electric-field electrode 310, and the second ultrasound electrode 320 may be provided in one or more numbers.

In one embodiment, the first electrode array 200 and the second electrode array 300 may include a ferroelectric material. The first electrode array 200 and the second electrode array 300 may include at least one of PZT(Pb[ZrₓTi₁₋ₓ]O₃), PMN-PT(Pb(MₓNb_{y})O₃-PbTiO₃), PMN-PZT(Pb(Mg_{1/3}Nb_{2/3})O₃-PbZrO₃-PbTiO₃), BaTiO₃. The first electrode array 200 and the second electrode array 300 may be formed of the ferroelectric material and thus may exhibit a piezoelectric effect.

FIG. 2 is a conceptual diagram illustrating a first electrode array, a second electrode array, and an affected region according to some embodiments. FIG. 2 will be described together with FIG. 1.

In one embodiment, an electric field EF may be formed between the first electrode array 200 and the second electrode array 300 by an AC voltage. A direction of the electric field EF may continuously change according to an amplitude and a frequency of the AC voltage. The electric field EF may have a frequency in a range of 10 kHz to 500 kHz.

In one embodiment, cell division of cancer cells TC may be inhibited by the electric field EF. The electric field EF may have a frequency that affects cellular functions of the cancer cells. The frequency of the electric field EF may target a cell division cycle of the cancer cells.

In one embodiment, ultrasound UF may be formed between the first electrode array 200 and the second electrode array 300 based on an AC voltage and piezoelectric elements. A frequency and an intensity of the ultrasound UF may continuously change according to a frequency and a magnitude of the AC voltage.

In one embodiment, permeability of a blood-brain barrier (BBB) may be increased by the ultrasound UF, thereby enhancing efficiency of access of a drug to the cancer cells TC. Cell division of the cancer cells TC may also be inhibited by the ultrasound UF. The ultrasound UF may have a frequency for opening the blood-brain barrier BBB. The ultrasound UF may have a frequency that affects cellular functions of the cancer cells.

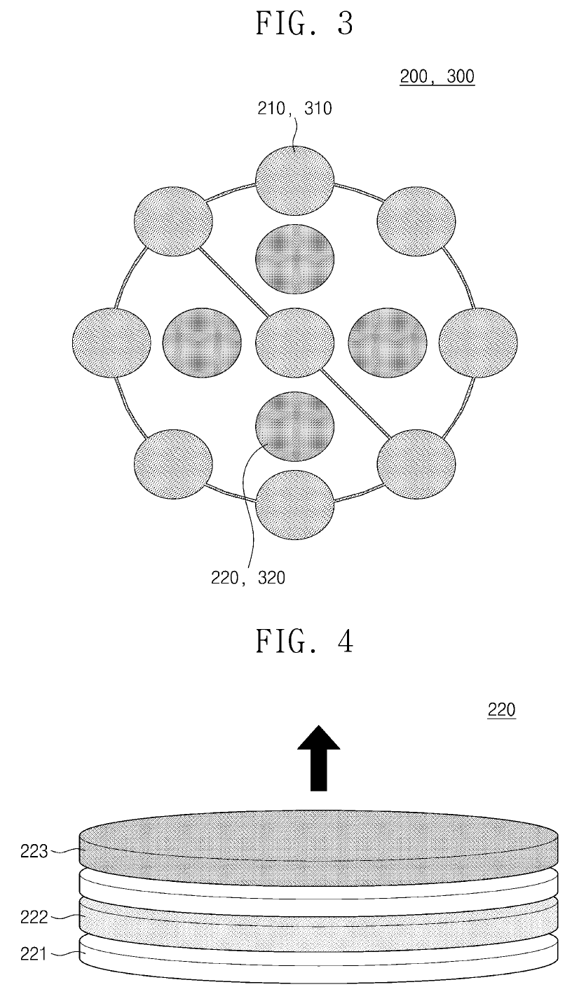

FIG. 3 illustrates an arrangement structure of electric-field electrodes and ultrasound electrodes included in the first electrode array and the second electrode array of FIGS. 1 and 2.

The first electrode array 200 and the second electrode array 300 may include electric-field electrodes 210 and 310, and ultrasound electrodes 220 and 320. By arranging the electric-field electrodes and the ultrasound electrodes in a mixed manner in the first electrode array 200 and the second electrode array 300, targeting efficiency of a drug to cancer cells may be enhanced.

In one embodiment, the ultrasound electrodes 220 and 320 may be configured as radially shaped multi-channel electrodes. The ultrasound electrodes 220 and 320 may be arranged to target the cancer cells TC.

In one embodiment, the electric-field electrodes 210 and 310 may include one electrode disposed at a center and a plurality of electrodes circularly arranged around the center. The electric-field electrodes 210 and 310 may be arranged according to a location at which the cancer cells occur.

FIG. 4 illustrates a structure of a first ultrasound electrode included in the first electrode array of FIGS. 1 and 2. A second ultrasound electrode 320 of the second electrode array 300 may have the same structure as the first ultrasound electrode 220 of the first electrode array 200.

Referring to FIG. 4, the first ultrasound electrode 220 of the first electrode array 200 may include a metal plate 221, a piezoelectric element 222, and a vibration plate 223.

In one embodiment, the metal plate 221 may be disposed above and below the piezoelectric element 222. The ultrasound generator 120 may apply an AC voltage to the metal plate 221. The ultrasound generator 120 may determine a frequency of the ultrasound by changing a frequency of the AC voltage, and may determine an intensity of the ultrasound by changing a magnitude of the AC voltage. The metal plate 221 may apply an electrical signal to the piezoelectric element 222.

In one embodiment, a material of the metal plate 221 may include a metal electrode such as gold (Au), silver (Ag), nickel (Ni), or aluminum (Al).

In one embodiment, a material of the piezoelectric element 222 may include PZT (lead zirconate titanate) or BaTiO₃ (barium titanate). The piezoelectric element 222 may vibrate by an electric field upon receiving an AC voltage, and may generate ultrasound. A wavelength of the ultrasound may be determined according to a thickness of the piezoelectric element 222.

In one embodiment, the vibration plate 223 may be disposed on a surface from which the ultrasound is emitted. The vibration plate 223 may protect the first ultrasound electrode 220 the piezoelectric element 222 from external impact, moisture, and chemical damage. The vibration plate 223 may reduce a difference in acoustic impedance between the piezoelectric element 222 and a medium. The vibration plate 223 may improve resolution and sensitivity of the generated ultrasound. A material of the vibration plate 223 may include a polymer (PMMA or silicone), a ceramic composite, or an elastomer (epoxy-based materials).

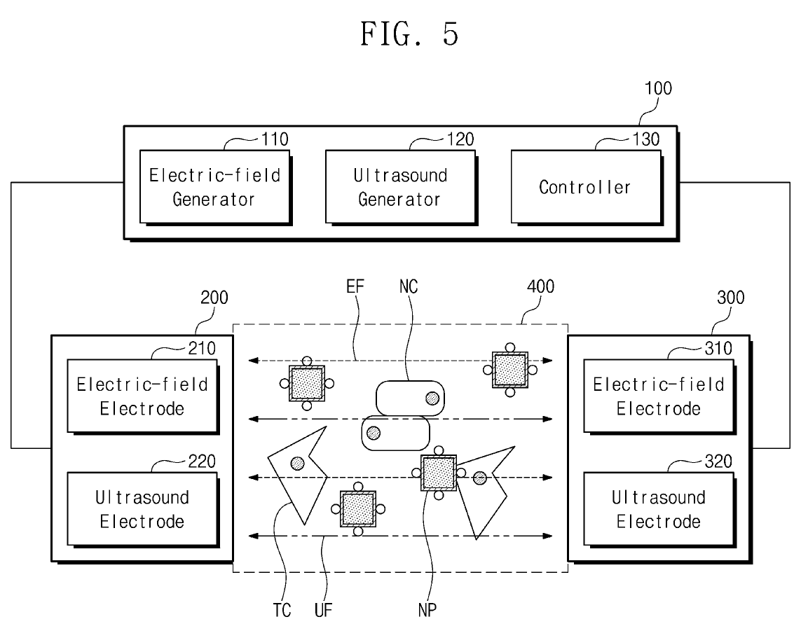

FIG. 5 illustrates a case in which the cancer treatment apparatus according to an embodiment of the present disclosure is used together with nanoparticle probes. A signal generator 100, a first electrode array 200, and a second electrode array 300 may perform the same functions as the signal generator 100, the first electrode array 200, and the second electrode array 300 of FIG. 1.

In one embodiment, an electric field EF may be formed between the first electrode array 200 and the second electrode array 300 by an AC voltage. Ultrasound UF may be formed by applying an AC voltage at a specific frequency between the first electrode array 200 and the second electrode array 300.

In one embodiment, a plurality of nanoparticle probes NP may be introduced into and/or around the cancer cells TC. The nanoparticle probes NP may enter an interior of a body through ingestion and may move to the affected region 400.

In one embodiment, the electric field EF may be applied to the nanoparticle probes NP. Polarization may occur within ferroelectric particles of the nanoparticle probes NP based on the electric field EF applied to the nanoparticle probes NP. That is, depending on a direction of the electric field EF, one portion of the ferroelectric particle may be charged positively (+) and another portion may be charged negatively (−).

A direction of the electric field EF may continuously change based on the AC voltage. Due to material properties, once polarization occurs in the ferroelectric particle, the polarized state may be maintained even when an electric field is not applied. When an electric field in a different direction is applied in the polarized state, re-polarization may occur according to the direction of the applied electric field.

An orientation of the nanoparticle probes NP may be changed based on a change in the direction of the electric field EF. During a process of changing the electric field EF, the ferroelectric particles may receive an electric force. Cell division of the cancer cells TC may be inhibited based on the change in the orientation of the nanoparticle probes NP.

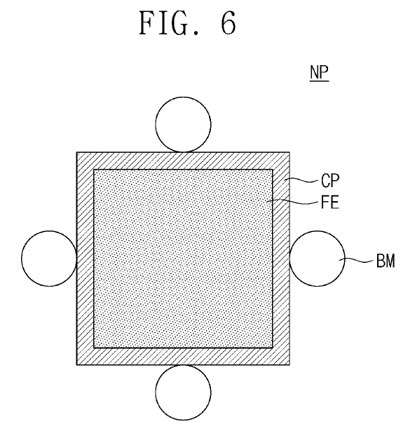

FIG. 6 illustrates a configuration of a nanoparticle probe NP. Referring to FIG. 6, the nanoparticle probe NP may include a ferroelectric particle FE and a plurality of biomarkers BM. If necessary, each of the nanoparticle probes NP may further include a non-dielectric insulating layer CP on a surface thereof.

In one embodiment, the ferroelectric particle FE may have a diameter ΔD greater than 0 nm and less than or equal to 100 nm. For example, the diameter ΔD of the ferroelectric particle FE may be greater than 1 nm and less than or equal to 100 nm. The ferroelectric particle FE may have a polygonal shape. The smaller the diameter ΔD of the ferroelectric particle FE, the more a permeability of a blood-brain barrier of the nanoparticle probe NP may be improved, as long as ferroelectric properties are maintained. The ferroelectric particle FE may be synthesized through a solvothermal synthesis method using barium hydroxide (Ba(OH)₂) and titanium butoxide (Ti[O(CH₂)₃CH₃]₄) as precursors. The ferroelectric particle FE may be formed of BaTiO₃ (barium titanate).

In one embodiment, the biomarkers BM may be attached to the nanoparticle probe NP. The nanoparticle probe NP may bind to receptors on a surface of cancer cells TC through the biomarkers BM. The nanoparticle probe NP bound to the receptors of the cancer cells TC may remain in such a state, or may be introduced into the cancer cells TC by phagocytosis of the cancer cells TC.

In one embodiment, the biomarkers BM may include at least one of transferrin and folate. The nanoparticle probe NP may target cancer cells by the biomarkers BM.

In one embodiment, the non-dielectric insulating layer CP may cover a surface of the ferroelectric particle FE. The non-dielectric insulating layer CP may be formed by placing the ferroelectric particle FE in a TEOS solution (Si(OC₂H₅)₄). The non-dielectric insulating layer CP may prevent non-specific binding reactions of the nanoparticle probe NP. The non-dielectric insulating layer CP may facilitate attachment of the biomarkers BM. The non-dielectric insulating layer CP may prevent polarization induced by an external electric field from dissipating due to an external electrolyte. The non-dielectric insulating layer CP may amplify an intensity of an induced electric field formed in a local area.

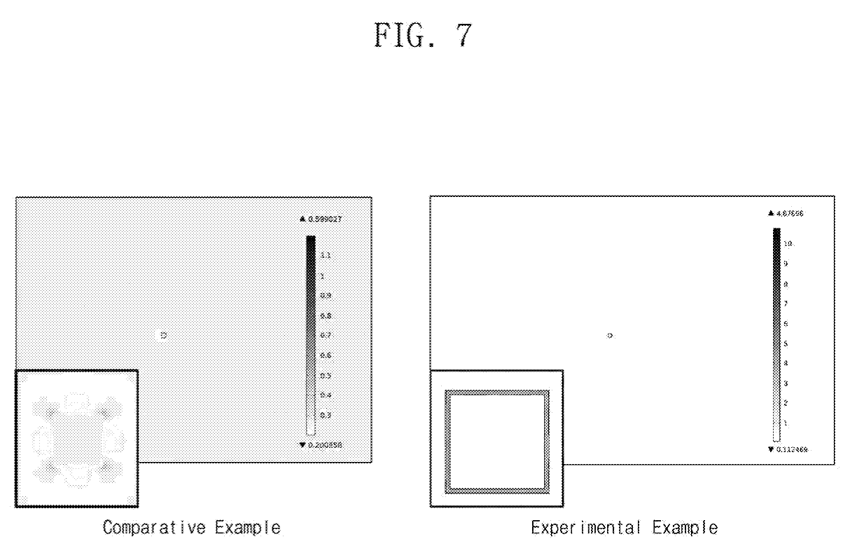

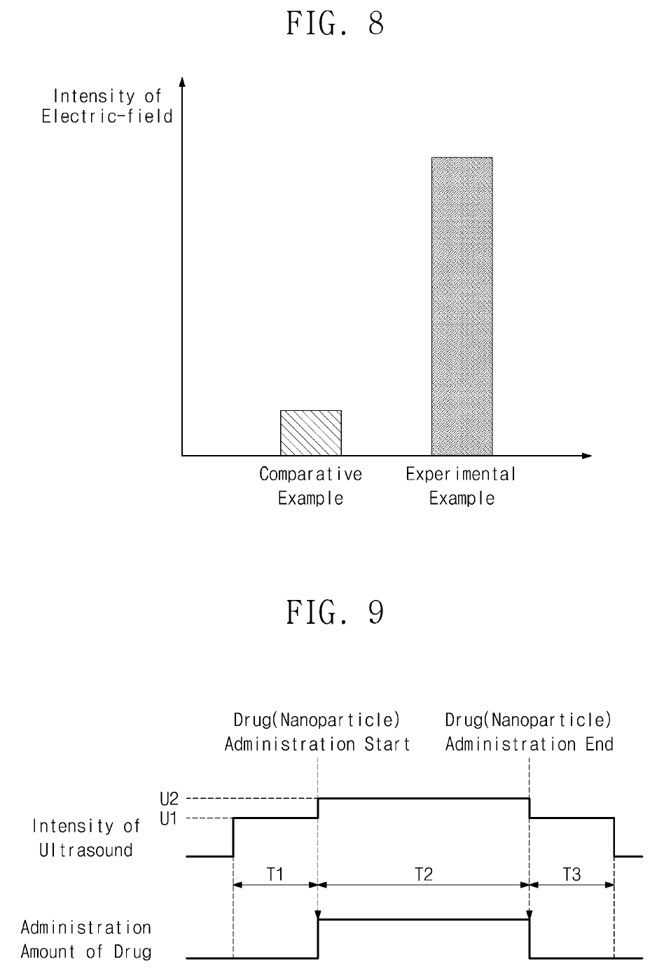

FIG. 7 is a comparison diagram between a comparative example without a silica layer (SiO₂) and an experimental example with a silica layer (SiO₂). FIG. 8 is a graph illustrating an electric-field intensity in the experimental example with a silica layer (SiO₂) compared to the comparative example without a silica layer (SiO₂).

### Comparative Example

PZT (lead zirconate titanate) electrodes were disposed at a distance of 24 mm from each other. A voltage of 10 V was applied between the electrodes. A BaTiO₃ nano cube having a size of 120 nm and having no silica layer (SiO₂) was used.

### Experimental Example

An experiment was conducted under the same conditions as the comparative example except for presence of a silica layer (SiO₂), using the same electrodes at the same distance and applying the same voltage. A BaTiO₃ nano cube having a size of 120 nm and having a silica layer (SiO₂) with a thickness of 5 nm on a surface thereof was used.

Referring to FIGS. 7 and 8, it can be confirmed that an induced electric field having an intensity 7 to 8 times greater is formed in the experimental example having the silica layer (SiO₂) compared to the comparative example without the silica layer (SiO₂).

FIG. 9 is a schematic diagram illustrating a strategy for enhancing electric-field-based cancer treatment efficiency by adjusting an ultrasound intensity according to an administration amount of a drug including nanoparticle probes. FIG. 9 will be described together with FIG. 5.

In one embodiment, during a period T1 before the drug is administered, the signal generator 100 may apply a voltage to the first electrode array 200 and the second electrode array 300 with an ultrasound intensity set to U1. During a period T2 in which the drug is administered, the signal generator 100 may increase a magnitude of the voltage while setting the ultrasound intensity to U2. During a period T3 after completion of drug administration, the signal generator 100 may decrease the magnitude of the voltage while setting the ultrasound intensity back to U1.

In one embodiment, the controller 130 may differently adjust an ultrasound intensity for opening the blood-brain barrier BBB and an ultrasound intensity for a piezoelectric effect according to an administration time of the nanoparticle probes NP.

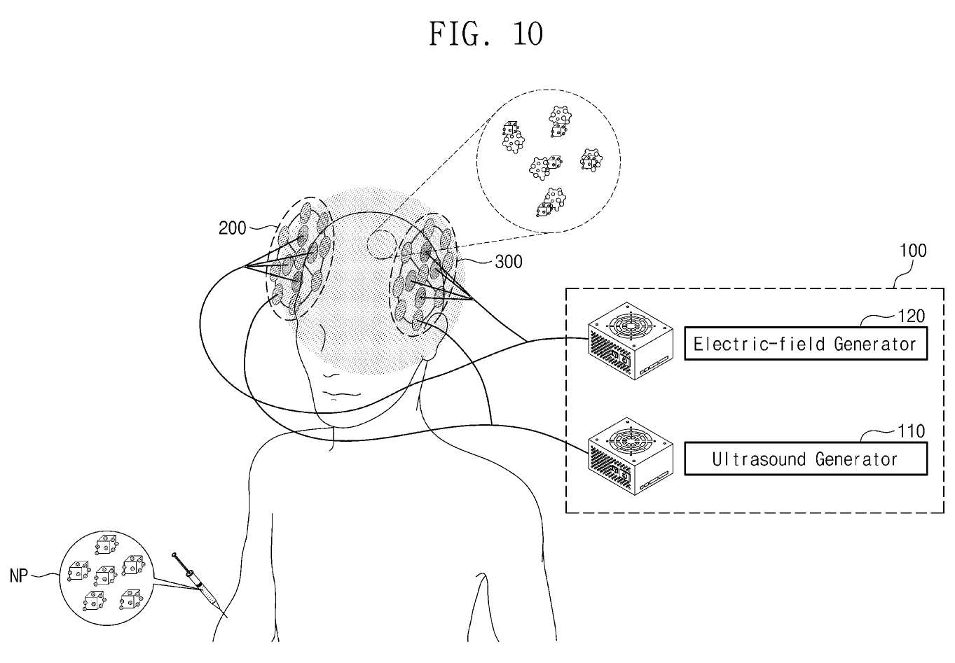

FIG. 10 illustrates an example of use of the cancer treatment apparatus according to the present disclosure.

Referring to FIG. 10, a plurality of nanoparticle probes NP may be introduced into and/or around cancer cells TC. The nanoparticle probes NP may enter an interior of a body through ingestion or injection and may move to the affected region. The first electrode array 200 and the second electrode array 300 including electric-field electrodes and ultrasound electrodes may be detachably provided on a head portion in which cancer cells are present.

As described in FIGS. 1-9, the first electrode array 200 and the second electrode array 300 may be disposed to be spaced apart from each other with the affected region interposed therebetween. An electric field and ultrasound may be generated from the electric-field generator 110 and the ultrasound generator 120 of the signal generator 100 electrically connected to the first electrode array 200 and the second electrode array 300. Based on the electric field and the ultrasound, the nanoparticle probes NP may more easily access the cancer cells, and therapeutic efficiency may be enhanced.

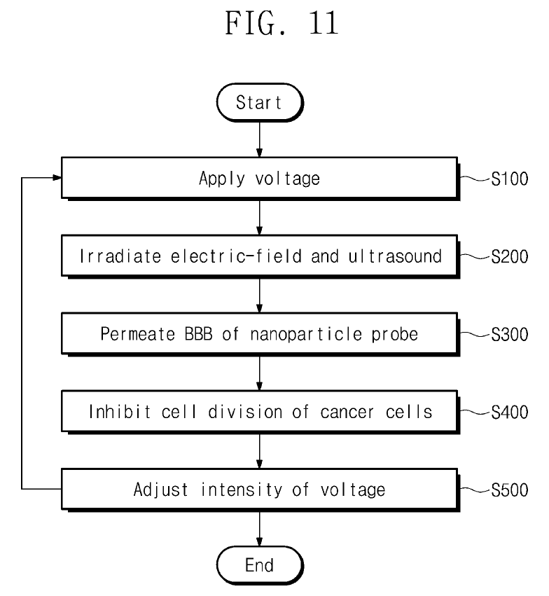

FIG. 11 is a flowchart illustrating an operation method of the cancer treatment apparatus according to the present disclosure. FIG. 11 will be described together with FIG. 5.

Referring to FIGS. 5 and 11, in step S100, the signal generator 100 may apply a voltage to the first electrode array 200 and the second electrode array 300. In step S200, the first electrode array 200 and the second electrode array 300 may irradiate an electric field and ultrasound to the affected region 400. In step S300, the nanoparticle probes NP may pass through a blood-brain barrier BBB based on the ultrasound. In step S400, the nanoparticle probes NP may inhibit cell division of the cancer cells TC. In step S500, the controller 130 may adjust an intensity of the voltage applied to the first electrode array 200 and the second electrode array 300 based on an administration time of the nanoparticle probes NP.

Although embodiments of the present disclosure have been described in detail above, the scope of the present disclosure is not limited thereto, and various modifications and improvements by those skilled in the art using the basic concept of the present disclosure also fall within the scope of the present disclosure.

The cancer treatment apparatus according to the present disclosure includes the nanoparticle probes, and may inhibit cell division of the cancer cells by increasing permeability and treatment efficiency of the nanoparticle probes based on the electric field and the ultrasound.

What is claimed is:

1. A cancer treatment apparatus comprising:
a signal generator; and
a first electrode array and a second electrode array electrically connected to the signal generator and facing each other with a target region containing cancer cells disposed therebetween,
wherein the signal generator generates an electric field and ultrasound between the first electrode array and the second electrode array, and
wherein each of the first electrode array and the second electrode array includes electric-field electrodes and ultrasound electrodes.

2. The cancer treatment apparatus of claim 1,
wherein the signal generator includes:
an electric-field generator configured to apply an AC (Alternating Current) voltage between the first electrode array and the second electrode array; and
an ultrasound generator configured to apply an AC voltage having a specific frequency between the first electrode array and the second electrode array.

3. The cancer treatment apparatus of claim 1,
wherein the signal generator includes a controller configured to adjust application amounts of the electric field and the ultrasound based on an administration time of a cancer treatment drug.

4. The cancer treatment apparatus of claim 1,
wherein the first electrode array is configured such that one electric-field electrode is disposed at a center and a plurality of electric-field electrodes are circularly arranged around the center, and ultrasound electrodes are radially disposed between the electric-field electrodes, and
wherein the second electrode array is configured such that one electric-field electrode is disposed at a center and a plurality of electric-field electrodes are circularly arranged around the center, and ultrasound electrodes are radially disposed between the electric-field electrodes.

5. The cancer treatment apparatus of claim 1,
wherein each ultrasound electrode included in the first electrode array and the second electrode array includes:
a metal plate configured to transmit an electrical signal;
a piezoelectric element configured to vibrate by the electrical signal; and
a vibration plate configured to protect the ultrasound electrode and the piezoelectric element.

6. A cancer treatment apparatus comprising:
a signal generator; and
a first electrode array and a second electrode array electrically connected to the signal generator and facing each other with a target region containing cancer cells disposed therebetween,
wherein the signal generator is configured to generate an electric field between the first electrode array and the second electrode array to change an orientation of nanoparticle probes within cancer cells,
wherein the signal generator is configured to generate ultrasound between the first electrode array and the second electrode array to enhance permeability of the nanoparticle probes through a blood-brain barrier, and
wherein each of the first electrode array and the second electrode array includes electric-field electrodes and ultrasound electrodes.

7. The cancer treatment apparatus of claim 6,
wherein the signal generator includes:
an electric-field generator configured to apply an AC (Alternating Current) voltage between the first electrode array and the second electrode array; and
an ultrasound generator configured to apply an AC voltage having a specific frequency between the first electrode array and the second electrode array.

8. The cancer treatment apparatus of claim 6,
wherein the signal generator includes a controller configured to adjust application amounts of the electric field and the ultrasound based on an administration time of the nanoparticle probes.

9. The cancer treatment apparatus of claim 6,
wherein the first electrode array is configured such that one electric-field electrode is disposed at a center and a plurality of electric-field electrodes are circularly arranged around the center, and ultrasound electrodes are radially disposed between the electric-field electrodes, and
wherein the second electrode array is configured such that one electric-field electrode is disposed at a center and a plurality of electric-field electrodes are circularly arranged around the center, and ultrasound electrodes are radially disposed between the electric-field electrodes.

10. The cancer treatment apparatus of claim 6,
wherein each ultrasound electrode included in the first electrode array and the second electrode array includes:
a metal plate configured to transmit an electrical signal;
a piezoelectric element configured to vibrate by the electrical signal; and
a vibration plate configured to protect the ultrasound electrode and the piezoelectric element.

11. The cancer treatment apparatus of claim 6,
wherein the nanoparticle probe includes:
a ferroelectric particle having a polygonal shape;
a non-dielectric insulating layer coated on the ferroelectric particle; and
a plurality of biomarkers attached to the non-dielectric insulating layer,
wherein the biomarkers target the cancer cells.

12. The cancer treatment apparatus of claim 11,
wherein the biomarkers of the nanoparticle probe include at least one of transferrin and folate.

13. The cancer treatment apparatus of claim 11,
wherein the ferroelectric particle has a diameter greater than 1 nm and less than or equal to 100 nm, and
wherein the ferroelectric particle is formed of BaTiO₃.

14. The cancer treatment apparatus of claim 11,
wherein the non-dielectric insulating layer includes at least one of silicon oxide (SiO₂), aluminum oxide (Al₂O₃), and silicon nitride (Si₃N₄14).

15. A method of operating a cancer treatment apparatus using ferroelectric nanoparticle probes, the method comprising:
applying a voltage to a first electrode array and a second electrode array included in the cancer treatment apparatus by a signal generator included in the cancer treatment apparatus; and
irradiating a target region containing cancer cells with an electric field and ultrasound by the first electrode array and the second electrode array.

16. The method of claim 15,
wherein the method further comprises adjusting an intensity of the voltage based on an administration time of the ferroelectric nanoparticle probes by a controller included in the cancer treatment apparatus.
